(19) United States
(12) Patent Application Publication
(10) Pub. No.: US 2012/0143279 A1
(43) Pub. Date: Jun. 7, 2012

(54) METHODS AND KITS FOR TREATING APPETITE SUPPRESSING DISORDERS AND DISORDERS WITH AN INCREASED METABOLIC RATE

(76) Inventors: Leon Ekchian, Glendale, CA (US); Alejandro Covalin, Los Angeles, CA (US)

(21) Appl. No.: 13/259,421

(22) PCT Filed: Mar. 24, 2010
(86) PCT No.: PCT/US10/28515

§ 371 Date: Feb. 24, 2012

Related U.S. Application Data
(60) Provisional application No. 61/162,927, filed on Mar. 24, 2009.
(60) Provisional application No. 61/173,173, filed on Apr. 27, 2009.

(51) Int. Cl. A61N 1/36

(52) U.S. Cl. 607/45

(57) ABSTRACT

Disclosed herein are kits and methods for treating appetite suppressing disorders and disorders with an increased metabolic rate by neuromodulation. A method of treating an appetite suppressing disorder or a disorder with an increased metabolic rate in a patient may include identifying the brain structure that is subject to modulation in the patient; and modulating the activity of one or more brain structures by applying electrical stimulation to one or more brain structures of a patient. A kit may include: a neuromodulation device; and instructions for using the neuromodulation device to modulate activity of a brain structure by applying electrical stimulation to one or more brain structures of a patient for treatment of an appetite suppressing disorder or a disorder with an increased metabolic rate.

### CROSS REFERENCE TO RELATED APPLICATIONS

This application claims the benefit of priority to U.S. Provisional Patent Application No. 61/162,927, which was filed Mar. 24, 2009 and is entitled “Methods for Treating Cachexia or Anorexia Via Deep Brain Stimulation,” and U.S. Provisional Patent Application No. 61/173,173 which was filed Apr. 27, 2009 and is entitled “Methods for Treating Cachexia or Anorexia Via Deep Brain Stimulation,” both of which are incorporated by reference herein in their entirety.

This application is also related to U.S. patent application Ser. No. 12/411,710 which was filed Mar. 26, 2009 and is entitled “Methods for Identifying and Targeting Autonomic Brain Regions,” which claims priority to U.S. Patent Application No. 61/039,671, which was filed Mar. 26, 2008, and is entitled “Methods for Identifying and Targeting Autonomic Brain Regions,” which are both incorporated by reference herein in their entirety.

### FIELD

The present disclosure relates generally to methods of treating appetite suppressing disorders and/or disorders with an increased metabolic rate, such as cachexia and anorexia, via neuromodulation, such as deep brain stimulation

### BACKGROUND

Appetite-stimulating drugs have been used to treat appetite suppressing disorders and/or disorders with an increased metabolic rate, such as cachexia and anorexia, in patients to increase food intake, but the drugs have generally not had the desired effect on body weight. A recent study was conducted using a pharmacological therapy in rodents that blocked hypothalamic receptors involved in the control of the energy homeostasis system. For example, when the melanocortin receptors were blocked, cachexia was diminished, and muscle mass was preserved and food intake increased. However, because melanocortin receptors are found in numerous tissues in the body, this pharmacological approach also has several undesirable systemic effects and side effects, such as possible loss of antinflammatory effects.

There is a need in the art for a targeted method of treating appetite suppressing disorders and/or disorders with an increased metabolic rate (including, e.g., hypermetabolic disorders) such as cachexia and anorexia.

The present disclosure was developed against this backdrop.

### SUMMARY

Disclosed herein is a method of treating an appetite suppressing disorder or a disorder with an increased metabolic rate in a patient. In one embodiment, the method includes identifying the brain structure that is subject to modulation in the patient; and modulating the activity of one or more brain structures by applying electrical stimulation to one or more brain structures of a patient, wherein the brain structure is chosen from the group consisting of the ventromedial hypothalamic nucleus, the perifornical region, the lateral hypothalamic area, the dorsomedial hypothalamic nucleus, the arcuate nucleus, and the paraventricular nucleus. In some embodiments, identifying the brain structure further comprises administering to the patient an effective amount of an agonist or an antagonist of a cellular receptor of the brain structure. In some embodiments, modulating the activity of a brain structure comprises modulating a system of the brain structure to treat an appetite suppressing disorder or a disorder with an increased metabolic rate.

In some embodiments, the system of the brain structure that is subject to modulation is chosen from the group consisting of the melanocortin system and the NPY system. In some embodiments, the method further comprises imaging the brain structure that is subject to modulation. In some embodiments, the method further comprises modulating the activity of one or more brain structures by chemical stimulation by administering to the patient an effective amount of an agonist or an antagonist of a cellular receptor of the brain structure. In some embodiments the appetite suppressing disorder is chosen from the group consisting of cachexia and anorexia.

In some embodiments, the brain structure is the ventromedial hypothalamic nucleus. In some embodiments, the brain structure is the ventromedial hypothalamic nucleus, the system is the melanocortin system and the cellular receptor is chosen from the group consisting of MCr3 and MCr4. The antagonist may be selected from the group consisting of PG901 and MCLO129. In some embodiments, the brain structure is modulated at high frequency stimulation or very high frequency stimulation. In some embodiments, the brain structure is selected from the group consisting of the dorsomedial portion of the ventromedial hypothalamic nucleus and the medial portion of the ventromedial hypothalamic nucleus. In some embodiments, identifying the brain structure that is subject to modulation further comprises administering glucose to the patient.

In some embodiments, the brain structure is the paraventricular nucleus. In some embodiments, the brain structure is the paraventricular nucleus, the system is the melanocortin system and the cellular receptor is chosen from the group consisting of MCr3 and MCr4. The antagonist may be selected from the group consisting of PG901 and MCLO129. In some embodiments, the brain structure is modulated at a high frequency stimulation or a very high frequency stimulation.

In some embodiments, the brain structure is the dorsomedial hypothalamic nucleus. In some embodiments, the brain structure is the dorsomedial hypothalamic nucleus, the system is the NPY system and the cellular receptor is an NPY receptor. The agonist may be selected from the group consisting of human/rat neuropeptide Y(2-36), dexamethasone[8] and N-acetyl[Leu 28, Leu 31] NPY (24-36). In some embodiments, the brain structure is modulated at very low frequency stimulation, low frequency stimulation or medium frequency stimulation.

In some embodiments, the brain structure is the lateral hypothalamic area. In some embodiments, the cellular receptor is selected from the group consisting of 5-HT2C receptor and MOR receptor.

In some embodiments, the method further comprises a step of fine-tuning the identification of the brain structure, wherein the step of fine-tuning comprises monitoring at least one of oxygen consumption, energy expenditure, carbon dioxide production or respiratory quotient. In some embodiments, the step of fine-tuning comprises monitoring oxygen consumption.

Disclosed herein is a kit. In some embodiments, a kit may comprise: a neuromodulation device; and instructions for using the neuromodulation device to modulate activity of a brain structure by applying electrical stimulation to one or more brain structures of a patient for treatment of an appetite suppressing disorder or a disorder with an increased metabolic rate. In some embodiments, the neuromodulation device comprises an implantable pulse generator, at least one lead and an extension. In some embodiments, the neuromodulation device is a deep brain stimulation system. In some embodiments, the appetite suppressing disorder is selected from the group consisting of cachexia and anorexia. In some embodiments, the one or more brain structures is selected from the group consisting of the ventromedial hypothalamic nucleus, the perifornical region, the lateral hypothalamic area, the dorsomedial hypothalamic nucleus, the arcuate nucleus, and the paraventricular nucleus. In some embodiments, modulating activity of a brain structure comprises modulating a system of the brain structure to treat an appetite suppressing disorder. The system may be selected from the group consisting of the melanocortin system and the NPY system.

In some embodiments, the instructions may further comprise identifying the brain structure to be modulated by administering to the patient an effective amount of an agonist or an antagonist of a cellular receptor of the brain structure. In some embodiments, the brain structure is the ventromedial hypothalamic nucleus. In some embodiments, the brain structure is the ventromedial hypothalamic nucleus, the system is the melanocortin system and the cellular receptor is chosen from the group consisting of MCr3 and MCr4. The antagonist may be selected from the group consisting of PG901 and MCLO129. In some embodiments, the brain structure is modulated at high frequency stimulation or very high frequency stimulation. In some embodiments, identifying the brain structure that is subject to modulation further comprises administering glucose to the patient. In some embodiments, the brain structure is a portion of the ventromedial hypothalamic nucleus selected from the group consisting of the dorsomedial portion of the ventromedial hypothalamic nucleus and the medial portion of the ventromedial hypothalamic nucleus.

In some embodiments, the brain structure is the paraventricular nucleus. In some embodiments, the brain structure is the paraventricular nucleus, the system is the melanocortin system and the cellular receptor is chosen from the group consisting of MCr3 and MCr4. The antagonist may be selected from the group consisting of PG901 and MCLO129. In some embodiments, the brain structure is modulated at a high frequency stimulation or a very high frequency stimulation.

In some embodiments, the brain structure is the dorsomedial hypothalamic nucleus. In some embodiments, the brain structure is the dorsomedial hypothalamic nucleus, the system is the NPY system and the cellular receptor is an NPY receptor. The agonist may be selected from the group consisting of human/rat neuropeptide Y(2-36), dexamethasone[8] and N-acetyl[Leu 28, Leu 31] NPY (24-36). In some embodiments, the brain structure is modulated at very low frequency stimulation, low frequency stimulation or medium frequency stimulation.

In some embodiments, the brain structure is the lateral hypothalamic area. In some embodiments, the brain structure is the lateral hypothalamic area and the cellular receptor is selected from the group consisting of 5-HT2C receptor and MOR receptor.

In some embodiments, the instructions further comprise a step of fine-tuning, wherein the step of fine-tuning comprises monitoring at least one of oxygen consumption, energy expenditure, carbon dioxide production or respiratory quotient. In some embodiments, the step of fine-tuning comprises monitoring oxygen consumption.

While multiple embodiments are disclosed, still other embodiments of the present disclosure will become apparent to those skilled in the art from the following Detailed Description, which shows and describes illustrative embodiments of the disclosure. As will be realized, the disclosure is capable of modifications in various aspects, all without departing from the spirit and scope of the present disclosure. Accordingly, the drawings and detailed description are to be regarded as illustrative in nature and not restrictive.

### BRIEF DESCRIPTION OF THE DRAWINGS

FIG. 1 shows the relationship between various hypothalamic nuclei.

FIG. 2 depicts a graph of stimulation amplitudes that may be used in aspects of the present disclosure.

### DETAILED DESCRIPTION

Appetite suppressing disorders and/or disorders with an increased metabolic rate include hypermetabolic conditions, cachexia and anorexia. Hypermetabolism is often present after trauma and infection including sepsis. For example, hypermetabolism is present after traumatic brain injury (TBI) and also after burns. In fact, the hypermetabolic response to burns is generally greater than the response seen from any other trauma or infection. AIDS patient with chronic infections also suffer from a hypermetabolic state as well as many cancer patients. A hypermetabolic response is also present in some autoimmune conditions such as in chronic obstructive pulmonary disease (COPD).

Patients with cachexia or anorexia may show signs of significant weight loss in addition to other symptoms. Cachexia is the loss of weight, muscle atrophy, fatigue, weakness and significant loss of appetite in someone who is not actively trying to lose weight. It can be a sign of various underlying disorders such that when a patient presents with cachexia, a doctor will generally consider the possibility of cancer, metabolic acidosis (from decreased protein synthesis and increased protein catabolism), certain infectious diseases (e.g. tuberculosis, AIDS), some autoimmune disorders (e.g. Crohn's disease, rheumatoid arthritis), chronic obstructive pulmonary disease (COPD), or addiction to drugs such as amphetamines or cocaine. Cachexia physically weakens a patient to a state of immobility stemming from loss of appetite, asthenia, and anemia. In many cases, these patients also suffer from a metabolic rate that is higher then what it is in a healthy individual with similar characteristics.

Cachexia occurs frequently with malignancy, is frequently seen in end-stage cancer and is associated with more than 20% of cancer related deaths. For example, patients with upper gastrointestinal cancer frequently suffer from substantial weight loss and patients with pancreatic cancer have an increased likelihood of developing a cachectic syndrome. Ghrelin levels are also high in patients who have cancer-induced cachexia.

Anorexia is a term for the general loss of appetite. As used herein, anorexia includes, but is not limited to, anorexia nervosa. It is frequently seen in patients with depression and malaise, along with the commencement of fevers and illnesses, in disorders of the alimentary tract (e.g. the stomach), and as a result of alcoholic excesses and drug addiction (e.g. cocaine).

Anorexia nervosa is a psychiatric illness that describes an eating disorder characterized by body image distortion, extremely low body weight and an obsessive fear of weight gain. Individuals with anorexia nervosa may attempt to control body weight through voluntary starvation, purging, excessive exercise or other weight control measures such as diet pills or diuretic drugs. While the condition primarily affects adolescent females, approximately 10% of people with the diagnosis are male. Anorexia nervosa, involving neurobiological, psychological, and sociological components is a complex condition that can lead to death in severe cases. Those suffering from the eating disorder anorexia nervosa also appear to have high plasma levels of ghrelin.

The energy homeostasis system, which includes the melanocortin system and thus the melanocortin receptors, is involved in the regulation of appetite and metabolic rate (also called energy expenditure or total energy expenditure). Inhibition of the melanocortin receptors by pharmacological methods has been shown to diminish cachexia, preserve muscle mass and increase food intake, but there are several undesirable systemic effects. The present disclosure relates to methods of inhibiting melanocortin receptors, such as by hypothalamic deep-brain stimulation, which may lessen the systemic effects of a solely pharmacological approach. In one embodiment, the method relates to reversibly disrupting the melanocortin cascade bilaterally at the ventromedial hypothalamic nucleus (VMH) via direct inhibitory neuromodulation using deep-brain stimulation (DBS). In some embodiments, the neuromodulation is carried out at mid-range to high frequencies (greater than approximately 150 Hz) or at very high frequencies (e.g. in the kHz range, e.g., 7 kHz).

Food intake can also be increased by modulation of the neuropeptide-Y (NPY) system. The present disclosure also relates to methods of exciting the neuropeptide-Y (NPY) system such as by hypothalamic deep-brain stimulation, which may lessen the systemic effects of a solely pharmacological approach. Methods of modulating feeding behavior and/or energy expenditure such as by hypothalamic deep-brain stimulation, which may lessen the systemic effects of a solely pharmacological approach are also provided.

### I. Neuromodulation

Neuromodulation may refer to a medical procedure in which the function of the nervous system is altered, such as for pain relief. Neuromodulation may include electrical stimulation, lesioning of a region of the nervous system, or pharmacotherapy. For example, deep brain stimulation (DBS), is a surgical treatment involving the implantation of a medical device which sends electrical impulses to specific parts of the brain. DBS directly modulates brain activity in a controlled manner. In various embodiments, its effects are reversible (unlike those of lesioning techniques).

Generally, the deep brain stimulation (neuromodulation) system includes three components: an implanted (implantable) pulse generator (IPG), a lead, and an extension. The IPG is a pulse generator used to stimulate excitable tissue such as nerve tissue. The IPG can be battery-powered or inductibly-powered or powered by a combination of a battery and inductibly-transmitted energy. IPGs are often encased in a biocompatible hermetic housing, such as a titanium case.

When an IPG is used to stimulate brain tissue, electrical pulses are delivered to the brain to modulate neural activity at the target site. The IPG may be calibrated by a neurologist, nurse or trained technician to optimize symptom suppression and control side effects. At its proximal end, the lead is electrically connected to the IPG either directly or via the extension. At its distal end, the lead is in contact with the target tissue via at least one electrode or contact point. In some embodiments, the lead may be a coiled wire insulated in polyurethane with four platinum iridium electrodes and is placed in the target area of the brain.

Various commercial IPGs may be used in various embodiments of the present disclosure. For example, commercial embodiments known in the art can be used. In certain embodiments, IPGs that can be used include the Medtronic Soletra or Kinetra IPGs (Medtronic, Minnesota), or Libra (St. Jude, Minnesota), used conventionally for DBS. Alternatively, a DBS developed to treat pain, such as the Restore and Restore Ultra IPGs (Medtronic, Minnesota), Eon, Eon mini, Renew, or Genesis (St. Jude, Minnesota), or Precision Plus (Boston Scientific Natick, MA) may be used. The IPG can be used for additional indications, including epilepsy (Responsive Neurostimulator system, Neuropace, Mountain View, Calif.), vagal neural signals (Maestro, Enteromedics St. Paul, Minn.), cochlear implants (Freedom, Cochlear Limited, Lane Cove, Australia), as well as other uses (Interstim II and Enterra, Medtronic, Minneapolis).

Examples of such DBS devices include, but are not limited to, devices designed for control of Parkinson's Syndrome, such as the Kinetra Model 7428 Neurostimulator or the Soletra Model 7426 Neurostimulators (Medtronic, Minnesota). The power source(s) generate electrical signals that are transmitted to the brain via extensions. Examples of such extensions include, for example, Model 7482 Extensions or two Model 7495 Extensions (Soletra), or either two Model 3387 DBS Leads or two Model 3389 DBS Leads. Other devices can be used for tremor control therapy. Examples of these devices include power sources therapy can include one single program Soletra Model 7426 Neurostimulator or one single program Model 7424 Itrel II Neurostimulator. The power source generates electrical signals that are transmitted to the brain via either one Model 7495 Extension or one Model 7482 Extension and either one Model 3387 DBS Lead or one Model 3389 DBS Lead. These components comprise the implantable portion of the Activa System for unilateral Activa Tremor Control Therapy (Medtronic, Minnesota).

In certain embodiments, the IPG is configured to deliver electrical stimulation at greater than 1 kHz, 2 kHz, 3 kHz, 4 kHz, 5 kHz, 6 kHz, 7 kHz, or 8 kHz.

In various embodiments, the IPG is configured to deliver DBS at one or more frequencies, or within a range of frequencies. The IPG can be configured to deliver electrical stimulation at frequencies less than, and/or greater than one or more of 50 Hz, 45 Hz, 40 Hz, 35 Hz, 30 Hz, 25 Hz, 20 Hz, 15 Hz, or 10 Hz. In various embodiments, the IPG can be configured to deliver electrical stimulation at frequencies greater than, and/or less than, one or more of 50 Hz, 60 Hz, 70 Hz, 80 Hz, 90 Hz, 100 Hz, 125 Hz, 150 Hz, 175 Hz, 200 Hz, 225 Hz, 250 Hz, 275 Hz, 300 Hz, 325 Hz, 350 Hz, 375 Hz, 400 Hz, 425 Hz, 450 Hz, 475 Hz, or 500 Hz. In various embodiments, the IPG can be configured to deliver electrical stimulation at a frequency greater than, and or less than, one of 500 Hz, 525 Hz, 550 Hz, 575 Hz, 600 Hz, 625 Hz, 650 Hz, 675 Hz, 700 Hz, 725 Hz, 750 Hz, 775 Hz, 800 Hz, 825 Hz, 850 Hz, 875 Hz, 900 Hz, 925 Hz, 950 Hz, or 975 Hz, or 1000 Hz. In various embodiments, the IPG can be configured to deliver electrical stimulation at greater and/or less than one or more of 1000 Hz, 2000 Hz, 3000 Hz, 4000 Hz, 5000 Hz, 6000 Hz, 7000 Hz, 8000 Hz, 9000 Hz, or 10000 Hz. In various embodiments, any of the above-referenced frequencies can be the upper or lower borders of an applied frequency.

The frequencies can be used for various embodiments. For example, depending on the particular neural system, lower frequencies tend to excite the neural elements (i.e. neural tissues), such as neurons, axons, dendrites, nerve endings and nerve bundles, while higher frequencies tend to preferentially excite axons and in some cases inhibit neurons, and even higher frequencies tend to inhibit all neural elements. By way of example but not limitation, low frequency electrical stimulation may be used to produce a net excitatory effect, or alternatively high frequency can be used to produce a net inhibitory effect. In additional non-limiting examples, low frequency electrical stimulation can be used to modulate the LHA and the Pe. High frequency electrical stimulation can be used to modulate the PVN and VMH in general, including but not limited to the dorsomedial portion of the VMH (dmVMH).

In various embodiments, the IPG is configured to deliver DBS via different waveforms. For example, square monophasic, square biphasic with or without charge balanced, sinusoidal, ramp, triangular, exponential, and/or any combination of theses waveforms.

In various embodiments, the IPG is configured to deliver DBS at a specific pulse width or range of pulse widths. The IPG can be configured to deliver pulse widths in the range greater than and/or less than one or more of 10 μs, 20 μs, 30 μs, 40 μs, 50 μs, 60 μs, 70 μs, 80 μs, 90 μs, 100 μs, 125 μs, 150 μs, 175 μs, 200 μs, 225 μs, 250 μs, 275 μs, 300 μs, 325 μs, 350 μs, 375 μs, 400 μs, 425 μs, 450 μs, 475 μs, 500 μs, 525 μs, 550 μs, 575 μs, 600 μs, 625 μs, 650 μs, 675 μs, 700 μs, 725 μs, 850 μs, 875 μs, 900 μs, 925 μs, 950 μs, 975 μs, 1000 μs, 1500 μs, 2000 μs, 2500 μs, or 3000 μs. Those of skill in the art will recognized that one or more of the above times can be used as border of a range of pulse lengths. Pulse lengths can be defined in terms of extremely short pulses (i.e. between 10 and 50 μs), short pulses (i.e. between 50 to 350 μs), medium width pulses (i.e. between 350 to 700 μs), long pulses (i.e. between 700 us to 1.5 ms), very long pulses (i.e. between 1.5 to 3 ms), and extremely long pulses (i.e. >3 ms). Without being limited to any mechanism or mode of action, in certain cases longer pulses can excite slower conducting neural elements such as smaller diameter axons, as well as neurons for a given amplitude, while shorter pulses can excite fast conducting neural elements such as big diameter axons.

In various embodiments, the IPG is configured to deliver DBS electrical stimulation at a range of voltage or current amplitudes, which in various embodiments can be voltage controlled, current controlled, or a combination of both (i.e., the IPG produces current controlled pulses as well as voltage controlled pulses). In other embodiments, the amplitude can be applied by a capacitive discharge. In various embodiments, the amplitude can be in a range greater than and/or less than one or more of 5 μA, 6 μA, 7 μA, 8 μA, 9 μA, 10 μA, 20 μA, 30 μA, 40 μA, 50 μA, 60 μA, 70 μA, 80 μA, 90 μA, 100 μA, 125 μA, 150 μA, 175 μA, 200 μA, 225 μA, 250 μA, 275 μA, 300 μA, 325 μA, 350 μA, 375 μA, 400 μA, 425 μA, 450 μA, 475 μA, 500 μA, 525 μA, 550 μA, 575 μA, 600 μA, 625 μA, 650 μA, 675 μA, 700 μA, 725 μA, 850 μA, 875 μA, 900 μA, 925 μA, 950 μA, 975 μA, 1 mA, 2 mA, 3 mA, 4 mA, 5 mA, 6 mA, 7 mA, 8 mA, 9 mA, 10 mA, 20 mA, 30 mA, 40 mA or 50 mA. Those of skill in the art will recognize that one or more of the above amplitudes can be used as a border of a range of amplitudes. Further, amplitudes can be described in terms of extremely low amplitudes (i.e. <10 uA and its equivalent voltage depending on the electrode(s)-tissue impedance), very low amplitudes (i.e. 10 to 100 uA and its equivalent voltage depending on the electrode(s)-tissue impedance), low amplitudes (i.e. 100 to 500 uA and its equivalent voltage depending on the electrode(s)-tissue impedance), medium amplitudes (i.e. 500 uA to 1 mA and its equivalent voltage depending on the electrode(s)-tissue impedance), high amplitudes (i.e. 1 mA to 5 mA and its equivalent voltage depending on the electrode(s)-tissue impedance), very high amplitudes (i.e. 5 mA to 10 mA and its equivalent voltage depending on the electrode(s)-tissue impedance), and extremely high amplitudes (i.e. >10 mA and its equivalent voltage depending on the electrode(s)-tissue impedance).

The actual amplitude can depend on several factors such as the distance between the electrode(s) and the target tissue, the distribution of the target tissue, the geometry of the electrode(s), the relative geometry and position between opposite and same polarity electrodes, the waveform, the actual polarity of the leading pulse, and other stimulation parameters such as frequency and pulse width. In order to reach a particular stimulation threshold (for a single neural element or for a given percentage of a population of neural elements such that a response is triggered), the amplitude, pulse width and frequency are not independent. In most cases the relationship between the amplitude, pulse width and frequency can be described by what is known in the art as strength-duration (S-D), strength-frequency (S-F), and strength-duration-frequency (S-D-F) curves, which can follow an exponential or hyperbolic mathematical form. The S-D-F curve is a 3 dimensional version of the better known 2 dimensional S-D and S-F curves.

In various embodiments, an electrode may be inserted at the brain region to allow the brain region to be identified at a later time for therapeutic treatment. For example, a lead that contains an electrode is implanted after targeting a specific brain region. The electrode remains at least until such a time as DBS is applied.

Stereotactic surgery may be used to place an electrode. After selecting a reference or the best available reference, the exact position of the target area is coded into 3D coordinates, which are then used to implant the electrode. The reference or best available reference can be chosen by using, for example, a stereotactic frame, a “frameless” stereotactic device, anatomical references or other appropriate reference. Stereotactic surgery works on the basis of three main components: 1) a stereotactic planning system, including atlas, multimodality image matching tools, coordinates calculator, etc, 2) a stereotactic device or apparatus and 3) a stereotactic localization and placement procedure. Stereotactic frame guidance and techniques, such as CT imaging, MRI targeting and microelectrode recording may be used to place chronic stimulating electrodes in the targeted area.

Modern stereotactic planning systems are computer based. The stereotactic atlas is a series of cross sections of anatomical structure (e.g. of the human brain), depicted in reference to a two-coordinate frame. Thus, each brain structure can be easily assigned a range of three coordinate numbers, which will be used for positioning the stereotactic device. In most atlases, the three dimensions are: latero-lateral (x), dorso-ventral (y) and rostro-caudal (z).

The stereotactic apparatus uses a set of three coordinates (x, y and z) in an orthogonal frame of reference (cartesian coordinates), or, alternatively, a polar coordinates system, also with three coordinates: angle, depth and antero-posterior location. The mechanical device has head-holding clamps and bars which puts the head in a fixed position in reference to the coordinate system (the so-called zero or origin). In small laboratory animals, these are usually bone landmarks which are known to bear a constant spatial relation to soft tissue. For example, brain atlases often use the external auditory meatus, the inferior orbital ridges, the median point of the maxilla between the incisive teeth. or the bregma (confluence of sutures of frontal and parietal bones), as such landmarks. In humans, the reference points, as described above, are intracerebral structures which are clearly discernible in a radiograph or tomogram.

Guide bars in the x, y and z directions (or alternatively, in the polar coordinate holder), fitted with high precision vernier scales allow the neurosurgeon to position the point of a probe (an electrode, a cannula, etc.) inside the brain, at the calculated coordinates for the desired structure, through a small trephined hole in the skull.

Currently, a number of manufacturers produce stereotactic devices fitted for neurosurgery in humans, as well as for animal experimentation. Examples of such stereotactic devices include, Leksell Stereotactic Frame (Elekta, Atlanta, Ga.), CRW Stereotactic Frame (Integra Radionics, Burlington, Mass.) (for human use), large and small animal Stoelting stereotactic frame (Stoelting Co., Wood Dale, Ill.), large and small animal Stereotactic Instruments (Harvard Apparatus, Holliston, Mass.). An example of a “frameless” stereotactic device or a device used in a “frameless” surgery includes VectorVision, made by BrainLAB of Westchester, Ill.

In various embodiments, at least one of the IPG and lead of the DBS system are surgically implanted inside the body. In some embodiments at least one burr hole, which size can be any size known in the art which allows the placement and fixation of the at least one lead positioning and anchoring the lead correctly. The electrode is inserted, with instrumental feedback and/or feedback from the patient for optimal placement. In some embodiments, one or more electrodes are unilaterally implanted. That is, one or more electrodes are implanted in a brain region on one side of the brain (e.g., one or more electrodes are implanted in the right VMH or in the left VMH). In other embodiments, the electrodes are bilaterally implanted. That is, one or more electrodes are implanted in a brain region on both sides of the brain (e.g., one or more electrodes are implanted in the right VMH and in the left VMH). In certain embodiments, the lead is connected to the IPG by the extension. In one embodiment, the extension is an insulated wire that runs from the head and down the side of the neck behind the ear to the IPG. In some embodiments it may be placed subcutaneously below the clavicle. In some embodiments, it may be placed subcutaneously behind the abdomen, in yet other embodiments where the IPG is cranially mounted the extension may be placed subcutaneously in the head.

DBS leads are placed in the brain according to the type of symptoms to be addressed. For example, in non-Parkinsonian essential tremor, the lead is placed in the ventrointermedial nucleus (VIM) of the thalamus. For the treatment of dystonia and symptoms associated with Parkinson's disease (rigidity, bradykinesia/akinesia and tremor), the lead may be placed in either the globus pallidus or subthalamic nucleus. As described in more detail below, the regions of the brain where an electrode may be placed for the treatment of cachexia and/or anorexia include the VMH, the PVN, the LHA, and the Pe. Methods of identifying these regions are also described in more detail below.

### II. The Energy Homeostasis System

The energy-homeostasis system includes both hypothalamic and extra-hypothalamic centers that are involved in processes regulating both the energy intake (E_{IN}) and the total energy expenditure (TEE). While E_{IN} has one component, food intake (F_{IN}), the TEE can be divided into two main components: the energy expended due to movement-related activities and the energy expended due to non-movement-related activities. This division is such that at any given time the sum of these two components is equal to the TEE. In various aspects, the movement-related energy expenditure can be the mechanical energy expenditure (MEE) and the non-mechanical energy expenditure (nMEE) as the difference between the TEE and the MEE (Harnack et al., Journal of Neuroscience Methods). In humans the nMEE represents up to 70% of the TEE (McClean et al, Animal and Human Calorimetry). The fact that body weight (BW) remains relatively constant is due to the proper regulation of the nMEE.

Several mutually interacting hypothalamic nuclei may influence the MEE by inducing a change in spontaneous locomotor activity (Castenada et al., Journal of Nutrition) and shivering thermogenesis (Thornhill et al., Canadian Journal of Physiology and Pharmacology). These same mutually interacting hypothalamic nuclei may also regulate both the F_{IN} and the nMEE through a net of complexly-interacting nuclei described below.

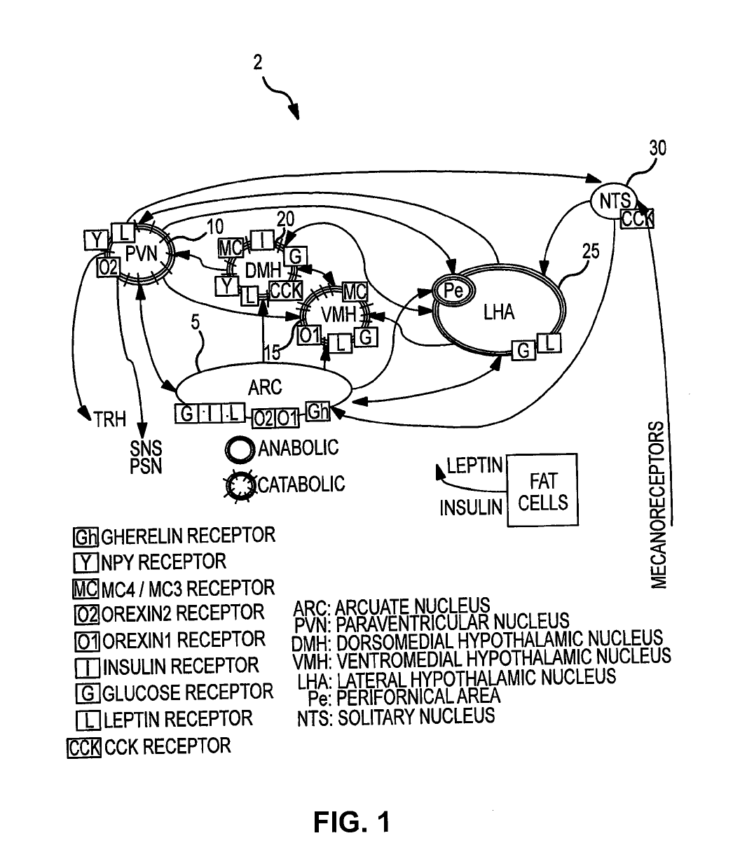

As can be understood from FIG. 1, at least five hypothalamic nuclei (or brain structures): Arcuate Nucleus (ARC) 5, Paraventricular (PVN) 10, Ventromedial Hypothalamic Nucleus (VMH) 15, Dorsomedial Hypothalamic Nucleus (DMH) 20 and the Lateral Hypothalamic Area (LHA) 25, may be involved in the regulation of the F_{IN} and the nMEE. Some of the afferent and efferent connections to and from these nuclei and their molecular mechanisms are known. In addition, at least part of the nMEE regulation may be exerted via sympathetic and parasympathetic modulation (Berthoud, Neuroscience and Biobehavioral Reviews). Indirect connections between hypothalamic nuclei and the vagus nerve via the nucleus of the solitary tract (NTS) may also provide signals that influence the F_{IN}.

a. The Arcuate Nucleus (ARC)

As shown in FIG. 1, the ARC 5, located at the inferior medial tuberal hypothalamic region, receives information from circulating molecules due to a leaky blood-brain-barrier in the area (at the median eminence) (Broadwell et al., Journal of Comparative Neurology), and from direct neuronal inputs. The ARC 5 may act as both an integrative center and a command center for the energy homeostasis system 2. In particular, signaling-molecules circulating in the blood are monitored to detect whether long-term energy (e.g. leptin), middle-term energy (e.g. insulin) and/or short-term energy (e.g. glucose and ghrelin) is available (Berthoud, Neuroscience and Biobehavioral Reviews; Bagnol, Current Opinion in Drug Discovery and Development). Generally, leptin, which is produced by adipose tissue, circulates in the blood stream in a concentration that is proportional to the amount of total body-fat tissue. Under abnormal circumstances, leptin concentration in the blood may be transiently uncorrelated to the total body-fat content (Kennedy et al., Journal of Clinical Endocrinology and Metabolism). The concentration of ghrelin, a hormone produced in the epithelial cells in the stomach (Wynne, Journal of Endocrinology), is at its lowest point after a meal, and the concentration level may increase until the next meal (Cowley, Neuron). The ARC 5 receives neuronal inputs from regions inside and outside the hypothalamus. Its intra-hypothalamic afferents originate mainly at the PVN 10, the LHA 25 and the VMH 15. Most of its extra-hypothalamic afferents originate at the NTS 30 (also known as the solitary nucleus), the amygdala, and the bed nucleus of the stria terminalis (Berthoud, Neuroscience and Biobehavioral Reviews; DeFalco et al., Science).

The ARC 5 may include at least two different neuronal populations that produce functionally antagonistic signaling molecules. One population produces pro-energy-conserving signaling molecules (ECm) and the other population produces pro-energy-expending signaling molecules (EEm). To regulate both F_{IN} and nMEE, these signaling molecules influence neuronal activity in other hypothalamic nuclei and in the ARC 5 (Williams et al., Physiology & Behavior). The pro-energy-conserving population produces neuropeptide-Y (NPY) and agouti gene-related peptide (AgRP), both of which possess potent energy-conserving effects (Hahn et al., Nature Neuroscience; Broberger, Proceedings of the National Academy of Sciences of the United States of America). The pro-energy-expending population produces pro-opiomelanocortin (POMC) and cocaine-and-amphetamine regulated transcript (CART) (Elias et al., Neuron; Kristensen, Nature). The POMC is a precursor to the α-melanocyte-stimulating hormone (α-MSH), and both the α-MSH and CART reduce F_{IN} and increase nMEE. The production of NPY/AgRP may be inhibited by NPY (NPY-Y2 receptor) (Broberger et al., Neuroendocrinology), α-MSH (ARC MC3 receptor) (Jobst et al., Trends in Endocrinology and Metabolism), leptin (Baskin et al., Journal of Histochemistry & Cytochemistry; Mercer et al., Journal of Neuroendocrinology), and insulin (Wang et al., Brain Research). The production of NPY/AgRP may be promoted by orexin (ORX) which is produced in the LHA 25 (Guan et al., Neuroreport; Horvath et al., Journal of Neuroscience; Peyron et al., Journal of Neuroscience), by ghrelin (Wynne, Journal of Endocrinology), and by circulating glucocorticoids (Williams et al., Physiology & Behavior). The production of POMC/CART may be decreased by α-MSH (ARC MC3 receptor) (Jobst et al., Trends in Endocrinology and Metabolism) and increased by leptin (Jobst et al., Trends in Endocrinology and Metabolism). However, medial VMH neurons, which may be directly or indirectly stimulated by POMC, send excitatory projections to POMC neurons in the ARC 5 (Sternson et al., Nature Neuroscience) thereby driving the melanocortin system.

The efferent pathways of these populations project mainly into other hypothalamic nuclei but also to extra-hypothalamic regions (Broberger et al., Proceedings of the National Academy of Sciences of the United States of America; Broberger et al., Physiology & Behavior). Efferent connections of the NPY/AgRP population project to the PVN 10, LHA 25, DMH 20, and VMH 15 (Berthoud et al., Neuroscience and Biobehavioral Reviews; Wynne et al., Journal of Endocrinology; Williams et al., Physiology & Behavior). Efferent connections to the POMC/CART population projects to the LHA 25 (e.g. into ORX producing neurons) (Elias et al., Neuron) and DMH 20 (e.g. NPY producing neurons). The POMC/CART-ARC neurons have direct projections to the VMH 15 (Wynne et al., Journal of Endocrinology; Guan et al., Molecular Brain Research) and the latter has numerous melanocortin receptors to which POMC binds (e.g. MC4R and MC3R) (Berthoud et al., Neuroscience and Biobehavioral Reviews; Bagnol et al., Current Opinion in Drug Discovery & Development; Wynne et al., Journal of Endocrinology).

In summary, the neuronal activity in the ARC 5 tends to balance the TEE and the F_{IN}. The ARC 5 monitors the energy status in the body and may act upon other hypothalamic nuclei in order to compensate for an imbalance in the energy system.

b. Paraventricular Nucleus (PVN)

The PVN 10 is located in the superior periventricular chiasmatic hypothalamic region. The PVN 10 is involved in several regulatory systems including the energy-homeostasis system. A decrease in the F_{IN} and an increase in nMEE, caused by excitatory electrical stimulation of the PVN 10, appears to be mediated by the potentiation of GABA-ergic interneurons. Afferent projections from the ARC 5 and from the DMH 20 that release NPY/AgRP and NPY respectively, inhibit GABA-releasing interneurons. The POMC/CART projections increase GABA release from the same interneurons into the PVN 10 (Cowley et al. Neuron). Other afferent projections into the PVN 10 originate at ORX-producing neurons in the LHA 25. These LHA-neurons may mediate their effect through the orexin receptor-2 (OX2R), which is abundant in the PVN 10 (Bagnol et al. Current Opinion in Drug discovery & Development). OX2R may modulate arousal in sleep-wakefulness cycles (Lin et al., Cell) but may not modulate F_{IN} because F_{IN} is affected by OXR acting upon OX1R (Lecea et al., Proceedings of the National Academy of Sciences of the United States of America; Haynes et al., Peptides). Non-endocrine efferents from the PVN 10 project to several hypothalamic nuclei, including the ARC 5, VMH 15, DMH 20, and LHA 25 (Terhorst et al., Brain Research Bulletin). Extrahypothalamic efferent projections from the PVN 10 terminate in the NTS 30 and in the preganglionic neurons. The projections that terminate in the NTS 30 trigger neuronal activity that exert an inhibitory effect in the dorsal motor nucleus (Zhang et al., American Journal of Physiology-Gastrointestinal and Liver Physiology). In turn, the dorsal motor nucleus has an excitatory effect on the autonomic nervous system (ANS) (Nishimura et al., Journal of Neurophysiology).

In summary, the PVN 10 receives inputs from and sends outputs to most hypothalamic nuclei involved in the energy-homeostasis system 2. The PVN 10 also projects to both sympathetic and parasympathetic neurons and thereby functioning as an integrating, processing, and actuating center for the energy-homeostasis system 2.

c. Ventromedial Hypothalamic Nucleus (VMH)

The VMH 15 is located in the medial tuberal hypothalamic region. The VMH 15 has been implicated in metabolic (Ruffin et al., Brain Research), reproductive (Nishimura et al., Journal of Neurophysiology), affective (Kruk, Neuroscience and Biobehavioral Reviews), and locomotor (Narita et al., Behav. Brain Res.) behavior. The VMH 15 may be anatomically divided into four regions that may be only slightly connected or completely unconnected. These four regions are the anterior VMH (aVMH), ventrolateral VMH (v1VMH), central VMH (cVMH), and dorsomedial VMH (dmVMH) (Canteras et al., Journal of Comparitive Neurology).

Within the energy-homeostasis system, the VMH 15 has been referred to as the “satiety center” (Schwartz et al., Nature). In addition, stimulation of the VMH may increase locomotor activity (Narita et al., Behav. Brain Res.), non-mechanical energy expenditure (nMEE), decrease F_{IN} (Ruffin et al., Brain Research), promote lipolysis (Ruffin et al., Brain Research; Takahashi et al. J. of the Autonomic Nervous System; Shimazu, Diabetologia), and stimulate non-shivering thermogenesis (Thornhill et al., Brain Research). Experiments have also shown that VMH activity may regulate glucose uptake in skeletal muscles during exercise (Vissing et al., American Journal of Physiology) and that lesions in the VMH 15 may produce obesity and hyperphagia (Williams et al., Physiology & Behavior). The activity in the VMH may be influenced by both short and long-term energy availability because it contains numerous leptin receptors (Shioda et al., Neuroscience Letters) and close to half of its neurons are stimulated by a glucose increase (Ashford et al., Pflugers Archiv-European Journal of Physiology; DunnMeynell et al., Brain Research; Muroya et al., Neuroscience Letters). In particular, the activity of the gluco-sensitive neurons in the VMH 15 is up-regulated by leptin and down-regulated by ORX (originating in the LHA) (Shiraishi et al., Physiology & Behavior).

The VMH 15 receives afferent projections from the ARC 5 (e.g. NPY/AgRP and POMC/CART neurons) (Wynne et al., Journal of Endocrinology), the LHA 25 (e.g. ORX and melanin-concentrating hormone neurons) (Jobst et al., Trends in Endocrinology and Metabolism), the DMH 20, the PVN 10, the ANH (Terhorst et al., Brain Research Bulletin), and the NTS 30 (Fulwiler et al., Neuroscience Letters). In addition to projecting efferent fibers to all of the above nuclei, the VMH 15 also projects to the PHA, the zona incerta (ZI), limbic areas, several thalamic nuclei, the amygdala, the periaqueductal gray, and to the entorhinal area (Canteras, et al., Journal of Comparative Neurology). Medial VMH neurons, which may be influenced by POMC produced by ARC neurons, send excitatory projections to POMC neurons in the ARC (Sternson et al., Nature Neuroscience) which may help to drive the melanocortin system.

In summary, the VMH 15 is anatomically divided and these divisions may be functionally different. With respect to the energy-homeostasis system, the VMH 15 integrates information about short-term and long-term energy availability and it may have functional connections with most of the other hypothalamic nuclei involved in the energy-homeostasis system. Thus, VMH activity may influence F_{IN}, MEE, nMEE, lipolysis, and glucose uptake in muscles.

d. Dorsomedial Hypothalamic Nucleus (DMH)

The DMH 20 is located in the medial tuberal region just dorsal to the VMH 15. Lesions in the DMH may cause changes in pancreatic-nerve activity (Elmquist et al., Proceedings of the National Academy of Sciences of the United States of America) and may induce hypophagia, thereby leading to a lower body weight (BW) (Bernardis et al., Proceedings of the Society for Experimental Biology and Medicine) and excitatory stimulation of the DMH may result in hyperglycemia (Elmquist et al., Proceedings of the National Academy of Sciences of the United States of America). These effects may be carried out via NPY-expressing neurons in the DMH that project to the PVN (Berthoud, Neuroscience and Biobehavioral Reviews).

From within the hypothalamus, the DMH 20 receives afferent projections from the VMH 15, the LHA 25, the ARC 5, and the anterior hypothalamic nucleus (AHN). From outside the hypothalamus, the DMH 20 may receive afferent projections from the periaqueductal gray, the hippocampal formation (e.g. ventral subiculum) and from the prefrontal cortex (Thompson et al., Brain Research Reviews). In addition, the DMH 20 may receive inputs for leptin and insulin receptors as well as from gluco-sensitive neurons expressed in the nucleus. The DMH 20 projects mainly to other hypothalamic nuclei, in particular to the PVN 10 but may also project to the VMH 15 and to the AHN, among others.

In summary, the DMH 20 may constitute an integrative center for intra- and extra-hypothalamic inputs that modulate aspects of the energy-homeostasis system, and such modulation may occur by influencing PVN 10 activity.

e. Lateral Hypothalamic Area (LHA)

The LHA 25 has extensive connections both inside and outside the hypothalamus. It sends and receives projections to and from the cortex, the thalamus, the basal ganglia, the mid-brain, the hippocampal formation, the NTS 30, and most hypothalamic regions (Berthoud, Neuroscience and Biobehavioral Reviews; Wynne et al., Journal of Endocrinology; Williams et al., Physiology & Behavior; Jobst et al., Trends in Endocrinology and Metabolism). In particular, information from the GI tract reaches the LHA 25 via the NTS 30 (Woods, AJP-Gastrointestinal and Liver Physiology).

The LHA may also receive information from circulation through leptin receptors (Elmquist, Neuroendocrinology of Leptin) and numerous gluco-sensing neurons that increase their firing rate in response to a decrease in circulating glucose (Ashford et al., Pflugers Archiv-European Journal of Physiology). In particular, a decrease in glucose may cause an increase in ORX production in the LHA 25 (Hakansson et al., Journal of Neuroendocrinology; Chemelli et al. Cell), which in turn may stimulate F_{IN} acutely (Bayer et al., Neuroreport). There are two types of ORX molecules produced in the LHA 25, Orexin-A (ORXa) and Orexin-B (ORXb) (Peyron et al., Journal of Neuroscience; Sakurai et al., Cell) and two receptors have been found to which ORX binds: OX1R and OX2R. The OX1R may have a much higher affinity (approximately 10-fold) for ORXa than for ORXb, while the other ORX receptor, OX2R, may have similar affinities for both ORXa and ORXb (Lund et al., Journal of Biological Chemistry). Experimental data suggests that only ORXa is directly related to the energy-homeostasis system. Intraventricular injections of ORXa may acutely promote feeding (de Lecea et al., Proceedings of the National Academy of Sciences of the United States of America; Haynes et al., Peptides), and blocking its effects with a specific antagonist may reduce F_{IN} (Yamada et al., Biochemical and Biophysical Research Communications). ORXb may play an important role in the arousal part of the sleep-wakefulness cycle, as shown by OX2R knockout-mice experiments in which the animals develop narcolepsy (Chemelli et al. Cell). In contrast to the VMH, where OX1R is heavily expressed, the PVN contains a substantial amount of OX2R (Bagnol, Current Opinion in Drug Discovery & Development). In the VMH 15, ORXa may inhibit the activity of gluco-sensitive neurons thus attenuating the response of the VMH 15 to an increase in the circulating glucose (Shiraishi et al., Physiology & Behavior). Experimental data suggests that both OX1R and OX2R are expressed in the ARC where they modulate, for example, NPY/AgRP and POMC/CART neurons (Burdakov et al., Journal of Neuroscience; Suzuki et al., Neuroscience Letters).

In summary, the LHA 25 receives information from many systems including the GI tract. The LHA 25 integrates information from all of these systems, and in turn, influences the expression of ECm and EEm in the ARC 5 as well as the glucose sensitivity in the VMH 15.

### III. Overview of Brain Region or Brain Structure Identification

Correctly identifying and targeting particular brain regions or brain structures during DBS is useful for successful medical intervention. One common technique to target brain regions or brain structures, e.g., neural structures (such as the hypothalamic nuclei disclosed herein), that are part of the central nervous system (CNS) and are functionally connected with the autonomic nervous system (ANS) and that have no unique or clearly identified direct correlation with human senses (i.e., vision, hearing, touch, smell, and taste) is via anatomical references. These anatomical references are derived via population studies and the anatomical location in a particular patient may be identified using magnetic resonance imaging (MRI) and comparing the MRI anatomical image with the above-mentioned anatomical references derived via population studies.

In the only human case using DBS targeting of hypothalamic structures (nuclei) that are related to the energy-homeostasis system and specifically the ventromedial hypothalamic nucleus (VMH), the target (i.e., the VMH) location was estimated using a computed tomographic scan (CT scan) (C. Hamani, et al., Ann. Neurology). The scan provided anatomical information to be used as a reference. After the electrode was at the estimated target, feedback from the patient was solicited for optimal placement of the electrode (i.e. subjective data). However, such targeting does not address variation in the locations of the brain regions or brain structures in different patients, and assumes that brain regions or brain structures are at the same location relative to other anatomical structures.

In additional embodiments, the present disclosure addresses the ability to identify brain regions or brain structures without referring to other anatomical locations for brain regions or brain structures that are part of the autonomic nervous system (ANS) and that have no unique or clearly identified direct correlation with our senses (i.e., vision, hearing, touch, smell, and taste).

In particular, the disclosure addresses the identification of deep brain structures that are the target in a deep-brain stimulation (DBS) paradigm. In some embodiments, the deep brain structures are hypothalamic structures (nuclei) with a known population of particular neurons which possess specific cellular receptors. Furthermore, the disclosure addresses the identification of hypothalamic structures involved in the energy homeostasis system. These structures may include sub-sets of the VMH, such as the dorsomedial portion of the VMH and the ventrolateral portion of the VMH, functional portions of the ventromedial hypothalamic nucleus (VMH). Additional structures may also include the perifornical region (Pe), the lateral hypothalamic area (LHA), the dorsomedial hypothalamic nucleus (DMH), the arcuate nucleus (ARC), and the paraventricular nucleus (PVN), in which neuronal activity is modulated by a particular agonist and/or inhibited by a particular antagonist many of which have a direct or indirect effect on energy expenditure, food consumption, glucose uptake in peripheral tissue, lipolysis, and other related functions of the energy homeostasis system, are identified and targeted.

An ordinarily skilled artisan will recognize that instead of using data obtained in a population study, the methods described herein may rely on data obtained before the surgery from each individual patient to fine-tune identification of the desired neural region(s) or structure(s), and confirm the placement of the electrode during the surgical procedure (intra-operatively) using objective measurements. For example, the methods disclosed herein may include monitoring at least one of oxygen consumption, energy expenditure, carbon dioxide production or respiratory quotient to fine tune identification of the desired neural region(s) or structure(s), and/or confirm the placement of the electrode during the surgical procedure (intra-operatively).

### IV. Identification of Brain Regions or Brain Structures

In certain aspects, the disclosure is also directed to methods of identifying one or more brain regions or brain structures. Brain structures may include the hypothalamic nuclei as described herein.

Any method of identifying or targeting a brain region or brain structure known in the art can be used. These include conventional methods of identifying a brain region or brain structure based on the relative position of the brain region or brain structure as compared to regions/structures of the patient's skull or other anatomical regions/structures. Conventional methods include those disclosed, for example, in Ting Guo, Andrew G. Parrent, and Terry M. Peters, “Automatic Target and Trajectory Identification for Deep Brain Stimulation (DBS) Procedures,” or Medical Image Computing and Computer-Assisted Intervention—MICCAI 2007, pages 483-490, both of which are incorporated by reference in their entireties. Alternatively, the methods used include methods of identifying a brain region or brain structure by administering a targeting agent that identifies a marker found at a brain region or brain structure, as described herein.

In certain aspects, the methods and apparatuses described herein can identify particular brain regions or brain structures using the receptors that are located on them, for example by the G-protein-coupled receptors. Since many brain regions or brain structures contain the same receptors, one way to identify a particular brain region or brain structure is by its unique or locally unique combination of receptors. For example, a method as described herein results in the direct or indirect stimulation or inhibition of the cells in the targeted brain region or brain structure such that the brain region or brain structure may be identified through at least one well-known functional imaging technique (e.g., fMRI, PET), and optionally, at least one well-known anatomical imaging technique (e.g., MRI, CT scan). Direct stimulation or inhibition can be done via at least one agonist or antagonist, respectively. Indirect stimulation or inhibition can be achieved using a secondary substance such that the activity in the targeted brain region or brain structure changes (i.e., stimulating or inhibiting the cells in the brain region); one of such examples would be ingesting glucose, which will, in a delayed manner, indirectly inhibit the activity in the VMH.

Recent publications have shown hyperactivity in the melanocortin system in cachexia (Laniano et al., American Journal of Physiology-Endocrinology and Metabolism; Inui, Ca-A Cancer Journal for Clinicians) as well as hypoactivity in the NPY system (Laviano, et al., Nutrition). Studies have shown that inhibiting the melanocortin system positively affects cachexia (Joppa et al., Peptides; Markison et al., Endocrinology; Foster et al., Idrugs; Flanagan et al., Brain Research). Experimental data shows that excitatory stimulation in the PVN may decrease F_{IN} by diminishing gastric motility (Flanagan et al., Brain Research); F_{IN} may be increased by increasing gastric motility.

Neural systems such as those described above may be modulated (inhibited or excited) via an electrical stimulation, as described herein. The present disclosure describes methods of identifying, targeting and modulating such neural systems. In some embodiments, the method of treatment includes inhibiting the melanocortin system. In some embodiments, the method of treatment includes exciting the NPY system. In some embodiments, the method of treatment includes modulation of feeding behavior and/or the energy expenditure. In still other embodiments, the method of treatment includes modulation of at least one of the melanocortin system, the NPY system and the feeding behavior and/or the energy expenditure of the patient.

In one embodiment, the nMEE is decreased and the food intake is increased via inhibition of the melanocortin system using high frequency DBS in the VMH, in particular in the medial portion of the VMH (mVHM) including the dorsomedial region of the VMH (dmVMH). In another embodiment, the nMEE is decreased and the food intake is increased via inhibition of the melanocortin system using very high frequency DBS in the VMH, in particular in the medial portion of the VMH (mVHM) including the dorsomedial region of the VMH (dmVMH). In another embodiment, feeding behavior is promoted by one or more frequencies administered at the LHA. In various embodiments, extremely low, very low, or low frequency excitatory stimulation can be administered to the LHA. In another embodiment, the nMEE is decreased and the food intake is increased via inhibition of the melanocortin system using high frequency DBS in the VMH, in particular in the medial portion of the VMH (mVHM) including the dorsomedial region of the VMH (dmVMH), and feeding behavior is further promoted via low frequency stimulation of the LHA. In another embodiment, the nMEE is decreased and the food intake is increased via inhibition of the melanocortin system using very high frequency DBS in the VMH, in particular in the medial portion of the VMH (mVHM) including the dorsomedial region of the VMH (dmVMH), and feeding behavior is further promoted via low frequency stimulation of the LHA and/or the Pe. In another embodiment, feeding behavior is promoted via medium range frequency stimulation of the PVN, in particular in the medial portion of the PVN.

Food intake can also be increased by modulation of the NPY system via very low frequency stimulation, low frequency stimulation, or medium frequency stimulation of the DMH. Food intake can also be increased by modulation of the NPY system via low frequency stimulation of the DMH. Exemplary melanocortin receptor antagonists include PG9O1 and MCLO129. Electrical stimulation can also be carried out via very high frequency DBS in the 3^{rd} ventricle to inhibit the medial VMH and/or the medial PVN. In other embodiments, a combination of the above-mentioned embodiments is used. In various other embodiments, the disorder or hypermetabolic condition can be treated chemically. For example, the brain regions or brain structures (such as the hypothalamic nuclei described herein) can be stimulated chemically via melanocortin antagonists delivered into the VMH and/or into the 3^{rd} ventricle. Alternatively, chemical stimulation can be carried out chemically via NPY agonists delivered into the DMH and/or into the 3^{rd} ventricle. Exemplary NPY agonists include human/rat neuropeptide Y (2-36), dexamethasone[8] and N-acetyl [Leu 28, Leu31] NPY (24-36).

The term “targeting agent” refers to an agonist or an antagonist of a cellular receptor found in a particular brain region and/or a substance that may indirectly activate or inhibit a particular brain region. Targeting agents can include any number of compounds known in the art (see, e.g., non-limiting examples provided in Tables 1-3 herein). In certain situations, the targeting agent specifically binds to a particular biological target, such as a particular receptor of a targeted brain region. The methods described herein are not limited to any particular targeting agent, and a variety of targeting agents can be used. The targeting agents can be, for example, various specific ligands, such as antibodies, monoclonal antibodies and their fragments, folate, mannose, galactose and other mono-, di-, and oligosaccharides, and RGD peptide. Other examples of such targeting agents include, but are not limited to, nucleic acids (e.g., RNA and DNA), polypeptides (e.g., receptor ligands, signal peptides, avidin, Protein A, and antigen binding proteins), polysaccharides, biotin, hydrophobic groups, hydrophilic groups, drugs, and any organic molecules that bind to receptors. When two or more targeting agents are used, the targeting agents can be similar or dissimilar. Utilization of more than one targeting agent can allow the targeting of multiple biological targets or can increase the affinity for a particular target. In some instances, the targeting agents are antigen binding proteins or antibodies or binding portions thereof. Antibodies can be generated to allow for the specific targeting of receptors of a particular brain region. Such antibodies include, but are not limited to, polyclonal antibodies; monoclonal antibodies or antigen binding fragments thereof; modified antibodies such as chimeric antibodies, reshaped antibodies, humanized antibodies, or fragments thereof (e.g., Fv, Fab′, Fab, F(ab′)2); or biosynthetic antibodies, e.g., single chain antibodies, single domain antibodies (DAB), Fvs, or single chain Fvs (scFv). Methods of making and using polyclonal and monoclonal antibodies are well known in the art, e.g., in Harlow et al, Using Antibodies: A Laboratory Manual: Portable Protocol I. Cold Spring Harbor Laboratory (Dec. 1, 1998). Methods for making modified antibodies and antibody fragments (e.g., chimeric antibodies, reshaped antibodies, humanized antibodies, or fragments thereof, e.g., Fab′, Fab, F(ab′)2 fragments); or biosynthetic antibodies (e.g., single chain antibodies, single domain antibodies (DABs), Fv, single chain Fv (scFv), and the like), are known in the art and can be found, e.g., in Zola, Monoclonal Antibodies: Preparation and Use of Monoclonal Antibodies and Engineered Antibody Derivatives, Springer Verlag (Dec. 15, 2000; 1st edition). In some instances, the targeting agents include a signal peptide. These peptides can be chemically synthesized or cloned, expressed and purified using known techniques. Signal peptides can be used to target brain regions as described herein.

During the target identification procedure, in order to increase the signal-to-noise ratio when the target area or region is being stimulated, the activity in areas surrounding the target area may be inhibited. Conversely, in order to increase signal-to-noise ratio when the target area is being inhibited, the activity in areas surrounding the target area could be stimulated. Stimulation and inhibition of any area can be done by using the appropriate targeting agents, such as agonists and antagonists.

Several imaging trials can be performed, each one using at least one agonist such that the target area can be identified by identifying the region that is commonly activated in all trials so that by superimposing the results of all trials the target region may be identified. Each trial involves a different agonist or antagonist that is common to the target region/area but are not all present in the surrounding regions/areas. In some embodiments, the images are superimposed via manipulation with computer software.

The particular targeting agents, e.g. agonist(s) and/or antagonist(s), are selected according to the desired target and its surrounding areas. Table 1 lists potential receptors that may be available in a respective target area.

TABLE 1

| Region | Potential Receptors |
|---|---|
| VMH (all) | Delta-opioid receptor (DOR) |
| | Cannabinoid receptor 1 (CB1) |
| | Corticotropin-releasing factor receptor 2 |
| | (CRF-R2) |
| | Kappa-opioid receptor (KOR) |
| | G-protein receptor 61 (GPR61) |
| | G-protein receptor 26 (GPR26) |
| | Glucocorticoid-induced receptor (GIR) |
| | Glucose (an agonist or antagonist of |
| | glucose is not needed, instead |
| | glucose itself is used.) |
| Dorsomedial | Orexin receptor 1 (OX1R) |
| portion | Orexin receptor 2 (OX2R) |
| of VMH | Melanocortin receptor 3 (MC3R) |
| | Neuropeptide Y receptor 5 (NPY-Y5R) |
| | Growth hormone-releasing hormone |
| | (GHRH) |
| | Melanin-concentrating hormone receptor |
| | 1 (MCHR1) |
| | Leptin |
| | Steroidogenic factor 1 (SF-1) |
| Ventrolateral | Melanocortin receptor 4 (MC4R) |
| portion | |
| of VMH | |
| Pe | KOR |
| | Mu-opioid receptor (MOR) |
| | MC4R |
| | MOR |
| | G-protein receptor 54 (GPR54) |
| LHA | Serotonin (5-HT 2C) |
| | MOR |
| | MCHR1 |
| DMH | KOR |
| | 5-HT |
| | MOR |
| | MCHR1 |
| | G-protein receptor 7 (GPR7) |
| | Prolactin releasing peptide receptor |
| | (PrRP-R) |
| | Glucagon-like peptide 1 receptor (GLP- |
| | 1R) |
| | Corticotropin-releasing factor receptor 1 |
| | (CRF-R1) |
| | GPR26 |
| | GPR54 |
| | Leptin |
| ARC | KOR |
| | Neuropeptide Y receptor 1 (NPY-Y1R) |
| | NPY-Y5R |
| | GHRH |
| | MC3R |
| | GLP-1R |
| | CRF-R1 |
| | GPR61 |
| | GPR26 |
| | GIR |
| | GPR54 |
| | Leptin |
| PVN | KOR |
| | NPY-Y1R |
| | NPY-Y2R |
| | NPY-Y5R |
| | MCHR1 |
| | MC4R |
| | GLP-1R |
| | CRF-R2 |
| | GPR61 |

As can be understood with reference to Table 1, for example, an ordinarily skilled artisan may use WIN 55212-2 (which is commercially available from vendors such as Perkin Elmer) as a CB1 agonist, [D-Trp8]-g-MSH as an MC3R agonist, RY764 as an MC4R agonist, PG9O1 as an MC3R antagonist, MCLO129 as an MC4R antagonist, etc. As another example, glucose can be ingested. Without being bound by mechanism, it may be likely that the action of glucose will be indirect, e.g., the activity in the VMH decreases when the glucose concentration in the blood is increased. Other agonists and antagonists of the receptors in Table 1 are well-known. In one embodiment used to identify the dorsomedial portion of the VMH, Orexin-1 (also called Orexin-a and hypocretin-a) may be intravenously administered. Orexin-1 can cross the blood-brain barrier and thus may serve as an agonist to OX1R.

After the targeting agents are selected, they may be administered to the patient. The administration can be done via many routes, for example, sub-cutaneous, intramuscular, intravenous, intracerebral ventricular, epidural, oral, or etc.

Functional Magnetic Resonance Imaging (fMRI) and/or Positron Emission Tomography (PET) can be used to image and measure the activity of the desired brain region or brain structure and of the entire brain. In some embodiments, temporal activity averaging technique such as Temporal Clustering Analysis (TCA) may also be used. Magnetic Resonance Imaging (MRI) and Computed Tomography Scan (CT scan) may also optionally be used to gather anatomical information. An ordinarily skilled artisan will recognize that when PET is used the agonists and antagonists should be altered so that they become radioactive isotopes.

An individualized activity map may be generated by combining images measuring the activity (e.g., several fMRI and/or several PET images) and anatomical images (e.g., MRI and/or CT-Scan). Given the introduction of agonist and/or antagonist, desired target area may be more active than its surroundings and thus identified and localized. For example, utilizing an agonist that activates the VMH along with utilizing an antagonist or antagonists that inhibit(s) the surrounding regions (e.g., the ARC and the DMH), generates a relatively higher activity signal arising from the VMH compared to utilizing the agonist alone.

In some embodiments, Temporal Clustering Analysis (TCA) may be used to interpret the data from the functional imaging techniques.

In some embodiments the modulation of the activity of the identified and targeted brain region is performed via implanted electrodes. In other embodiments, the modulation of the activity of the brain region is performed via local drug delivery. In other embodiments, the modulation of the activity is performed by a combination of implanted electrodes and local drug delivery. In yet other embodiments the modulation of the activity of the brain region is performed via non-invasive methods such as ultrasound, transcranial magnetic stimulation (TMS), and/or energy beams that can change the temperature in the target tissue.

When the target area is one that controls the overall metabolic rate and/or the percentage of oxidation of the main nutrients (i.e., carbohydrates, proteins, and fats), for example the dmVMH, indirect calorimetry may be used during the implantation procedure to fine tune the identification of the target. In this case, as the electrode approaches the coordinates of the target, calorimetry is performed. Since indirect calorimetry measures the energy expenditure (i.e., the metabolic rate) as well as the respiratory quotient (RQ), the indirect calorimetry measurements will reveal when the target is reached. After the target is reached, the electrode is fixed in place using standard neurosurgical techniques. The RQ changes when the oxidation-rate ratio carbohydrates/fats changes. The RQ decreases when the dorsomedial portion of the VMH is stimulated with excitatory low-intensity currents. Protocols for implementation or performance of indirect calorimetry are known. All references described herein are incorporated by reference in their entirety as if their contents were a part of the present disclosure.

### Kits

The present disclosure is also directed to kits that can be used to treat an appetite suppressing disorders and/or disorders with an increased metabolic rate in a patient. The kits can include a device, or component thereof, for delivery DBS to a patient. The kits can further include instructions for delivering deep brain stimulation to a patient. The kits may further include instructions for modulating the activity of a brain structure according to any of the methods disclosed herein, including but not limited to, modulating a system of the brain structure to treat an appetite suppressing disorder or a disorder with an increased metabolic rate.

In various embodiments, the kits include a device or component thereof for treating via DBS. The devices can include any commercial DBS system known. For example, one or more of the commercial IPGs (including, but not limited to IPGs described here) may be included in the kit. In alternative embodiments, any leads and/or electrodes may be used separately, or in combination with the IPG to form the system. In certain embodiments, the IPG may be designed to generate frequencies greater than or equal to 1.0 kHz, 2.0 kHz, 3.0 kHz, 4.0 kHz, 5.0 kHz, 6 kHz, 7 kHz, or 8 kHz.

The kits can further include instructions for treating an appetite suppressing disorder and/or disorders with an increased metabolic rate, including but not limited to cachexia and anorexia. In one embodiment, the kit may include: a neuromodulation device and instructions for using the neuromodulation device to modulate activity of a brain structure by applying electrical stimulation to one or more brain structures of a patient for treatment of an appetite suppressing disorder or a disorder with an increased metabolic rate. In one aspect, the neuromodulation device comprises an implantable pulse generator, at least one lead and an extension. In one aspect, the neuromodulation device is a deep brain stimulation system. In some aspects, the appetite suppressing disorder is chosen from the group consisting of cachexia and anorexia. In some aspects, the one or more brain structures is chosen from the group consisting of the ventromedial hypothalamic nucleus, the perifornical region, the lateral hypothalamic area, the dorsomedial hypothalamic nucleus, the arcuate nucleus, and the paraventricular nucleus. In some aspects, the modulating activity of a brain structure comprises modulating a system of the brain structure to treat an appetite suppressing disorder. The system may be chosen from the group consisting of the melanocortin system and the NPY system.

In some aspects, the brain structure is the ventromedial hypothalamic nucleus. The system is the melanocortin system and the cellular receptor is chosen from the group consisting of MCr3 and MCr4. The antagonist is selected from the group consisting of PG901 and MCLO129. The brain structure is modulated at high frequency stimulation or very high frequency stimulation. In some aspects, identifying the brain structure that is subject to modulation further comprises administering glucose to the patient. In some aspects, the brain structure is selected from the group consisting of the dorsomedial portion of the ventromedial hypothalamic nucleus and the medial portion of the ventromedial hypothalamic nucleus.

In some aspects, the brain structure is the paraventricular nucleus. The system is the melanocortin system and the cellular receptor is chosen from the group consisting of MCr3 and MCr4. The antagonist is selected from the group consisting of PG901 and MCLO129. The brain structure may be modulated at a high frequency stimulation or a very high frequency stimulation.

In some aspects, the brain structure is the dorsomedial hypothalamic nucleus. The system is the NPY system and the cellular receptor is an NPY receptor. The agonist is selected from the group consisting of human/rat neuropeptide Y(2-36), dexamethasone[8] and N-acetyl[Leu 28, Leu 31] NPY (24-36). The brain structure may be modulated at very low frequency stimulation, low frequency stimulation or medium frequency stimulation.

In still other aspects, the brain structure is the lateral hypothalamic area. The cellular receptor is chosen from the group consisting of 5-HT2C receptor and MOR receptor.

In various embodiments, the instructions may also provide directions for implanting a device into a patient, and for treating a patient with cachexia, anorexia or other disorder. The instructions can include any method disclosed herein for modulating the activity of a brain region by applying electrical stimulation to one or more brain regions or brain structures. The electrical stimulation can have any properties disclosed herein, including frequency, pulse width, amplitude, duration etc.

### EXAMPLES

The following examples are intended to be non-limiting and illustrative of aspects of the present disclosure.

### Example 1

Brain structures expressing MC3R and MC4R receptors, such as the VMH and the PVN, are used to modulate the melanocortin system to treat cachexia. In one embodiment where the effects of cachexia are to be mitigated, brain structures with MC3R and MC4R are identified and targeted. In particular, the VMH and PVN are targeted in order to inhibit the melanocortin system, which is hyperactive in cachexia. Inhibition is performed by high to very high frequency stimulation in the target region.

The patient is positioned in the MRI scanner and fMRI is continuously taken. The level of activity in the hypothalamus is measured using Temporal Clustering Analysis (TCA).

The patient drinks glucose with or without water. As the glucose concentration in the blood rises, the activity in the VMH decreases and therefore the area in the hypothalamus corresponding to the VMH can be identified as the brain structure where the activity is decreasing. Alternatively, the patient is administered a direct agonist of MC3R receptors. In this case the activity in the VMH increases and thus the VMH is identified as the brain structure where the activity in increasing. Once the VMH is identified, its coordinates are recorded using the best available reference. In some embodiments the reference is chosen by using a stereotactic frame.

### Example 2

In order to modulate neuronal activity, an electrode is surgically implanted into the dorsomedial portion of the VMH using the coordinates obtained as described above. In another experiment, a different technique to modulate the neuronal activity is used. An increase in energy expenditure (EE) and lipolysis is expected when excitatory stimulation is applied to the dorsomedial portion of the VMH specifically. Therefore, in order to fine tune the location and/or identification of the brain region, e.g., verify that the modulation is being applied to the desired targeted brain region, at least one of the following is monitored: a) the oxygen consumption (V02), b) the energy expenditure (EE), c) the carbon dioxide production (VCO2), and d) the respiratory quotient (RQ=VCO2NO2). In some experiments, EE, VO2, VCO2, and RQ is monitored using indirect calorimetry. Using indirect calorimetry, an increase in lipolysis is signaled by a decrease in the RQ.

In other embodiments, aside from the MC3Rs, the distribution of the MC4Rs is identified by performing steps 1-3 above using an appropriate MC4R agonist and the location of the best target determined to be such that a percentage of MC3Rs and a percentage of MC4Rs are modulated.

In some embodiments, PET and CT are used to identify the target. In these embodiments the appropriate agonists are made isotopes or paired with isotopes such that they can be detected using PET.

In still other embodiments, the ventrolateral portion of the VMH is identified in a method similar to that described above with respect to identification of the dorsomedial portion of VMH.

In still other embodiments, the PVN is identified in a method similar to that described above with respect to identification of the dorsomedial portion of VMH.

### Example 3

Brain structures expressing NPY receptors, such as the PVN region, are used to modulate the NPY system to treat cachexia. First, brain structures with NPY receptors are identified and targeted. In particular, the PVN may be targeted in order to modulate the NPY system, which is hypoactive in cachexia. Inhibitory-like stimulation of the target region are by medium range, high, or very high frequency stimulation.

The patient is positioned in the MRI scanner and fMRI is continuously taken. The level of activity in the hypothalamus is measured using Temporal Clustering Analysis (TCA).

The location of, for example, the PVN is functionally determined by administering, e.g., an agonist of the NPY receptor via, e.g., intravenous or intraderebroventricular injection. The patient is administered an agonist of the NPY receptor, the activity in the PVN increases and thus the particular area can be identified. Once the PVN is identified, its coordinates are recorded using the best available reference. In one embodiment, the reference is chosen using a stereotactic frame.

In order to modulate neuronal activity, an electrode is surgically implanted into the PVN using the coordinates as described above. In another experiment, a different technique to modulate the neuronal activity is used.

### Example 4

In other embodiments where the effects of cachexia are to be mitigated, brain structures that modulate food intake such as the LHA can be identified via the localization of 5-HT2C or MOR receptors and anatomical correlates targeted to increase food intake. Modulation or mitigation may then be carried out as described above.

### Example 5

An animal model is developed to study appetite suppressing disorders such as cachexia and anorexia. The animal model is used to monitor and analyze the progression of such disorders and may be used to study potential treatments of such disorders. Treatments include chemical treatments or electrical stimulation, such as deep brain stimulation.

a. General Overview

Male Lewis rats are used. Pancreatic cancer tumors are generated in donor rats by subcutaneously injecting a pancreatic adenocarcinoma cell line. Approximately eight weeks after the injection the donor animals are sacrificed and fragments of the generated tumors are implanted into the pancreas of a second group of tumor-recipient rats (n=24).

These tumor-recipient rats are randomly assigned to one of three groups (n=8). One group is used as a naive control (NC group), the other two groups receive bilateral DBS electrodes into the VMH. One of the VMH-implanted group serves as a sham control (SC group) and the other as the treatment group (TR group). Only the TR group is subjected to inhibitory neuromodulation treatment.

The tumor implantation procedure, which takes approximately 25 minutes and the DBS surgery, which takes approximately 90 minutes, are done in tandem. The animals are monitored at least two weeks before the implantation procedures, and are continually monitored until the end of the experiment. Each individual animal reaches the experimental endpoint when its body weight drops 20% or earlier as determined by a vivarium veterinarian. Animals are sacrificed by cardiac perfusion. General necropsy and histology of the tumor and the hypothalamic region containing the electrodes are performed.

Blood samples are taken from each animal once a week to assess general health and tested for the presence of several molecules related to the energy homeostasis system. Blood is drawn from the jugular vein under isoflurane anesthesia. The blood tests that are performed include: a metabolic panel, a lipid panel, insulin concentration, glycerol concentration, leptin concentration, ghrelin concentration and cholecystokinin concentration, as are known in the art.

The melanocortin system modulates TEE and Fᵢₙ and the system modulates cachexia-related symptoms. Thus, monitoring and analysis of certain molecules involved in the metabolic dysfunction generated by cancer cachexia is useful. Glucose (from the metabolic panel), leptin, insulin, ghrelin, modulate the melanocortin system partly via the ARC and the VMH (leptin and glucose only in the VMH). Cholecystokinin (CCK) interacts with the melanocortin system to regulate Fᵢₙ. Glycerol is used as a measure of lipolysis and triglyceride level from the lipid panel will be used as a measure of lipogenesis.

b. Development of an Animal Model

The animal model is designed such that metastasis does not occur too fast, allowing for a better time resolution to study the progression. This is achieved by confining the cancer cells to the pancreas.

Donor Animals: Rat ductal pancreatic adenocarcinoma cell line DSL-6A/C1 (American Type Culture Collection; Rockville, Md. U.S.A.) is cultured in Waymouth's MB 752/1 medium (Gibco, Grand Island, N.Y., U.S.A.) using procedures that are known in the art (e.g. Hotz et al., 2001). The donor animals are anesthetized (e.g. by isofurane) and the cultured cell is subcutaneously injected into both flanks of the animals. After eight weeks, donor animals are sacrificed by an overdose of sodium pentobarbital. Tumors are harvested under aseptic conditions and cut (e.g. by scalpel no. 11) into fragments, e.g., approximately 1 mm³ fragments. Macroscopically viable tumor tissue from the outer part of the tumors is used. Necrotic tissue from the central portion of the tumors is not used.

Recipient Animals: A group of 24 rats (275-325 g) are randomly assigned to one of the three above-described groups (n=8): the NC group, the SC group, and the TR group. Two weeks before implantation the animals are individually housed in metabolic chambers (e.g. CLAMS, Columbus Instruments, Columbus Ohio). While in the chambers, total energy expenditure (TEE), food intake (Fᵢₙ) and relative lipolysis is measured regularly. A relative lipolysis measure is obtained from the respiratory quotient (RQ). Also, blood samples are taken once or twice a week.

After two weeks, all animals are implanted with fragments of the tumors collected from the donor animals. An orthotopic implantation technique is used (e.g. Hotz et al., 2001). Briefly, the tumor-recipient rats are anesthetized (e.g. isoflurane) and kept under anesthesia with an anesthesia machine. A median incision under aseptic conditions at a laminar air flow working bench is made to open the abdomen of the animals and the spleen with the tail of the pancreas is exteriorized. In order to implant the tumor fragments, three tissue pockets are prepared in the pancreatic parenchyma. These pockets serve as implantation beds. One donor tumor fragment is placed into each one of the pockets such that tumor tissue is completely surrounded by pancreatic parenchyma. No sutures or glue are used to fix the tumor fragments to the recipient pancreas. The pancreas is reinserted and the initial incision is closed using absorbable sutures. The implantation procedure takes approximately 25 minutes.

After the surgery, the animals from the NC group are returned to the metabolic chambers. Animals in the SC and TR groups are placed in a stereotactic frame (e.g. Kopf instruments, Tujunga, California) and bilateral electrodes are implanted into the VMH.

c. Monitoring and Analysis of the Animal Model

The animal model is monitored and analyzed to study the progression of appetite-suppressing disorders such as cachexia and anorexia.

Total Energy Expenditure (TEE) is assessed. Indirect calorimetry is used to assess the TEE. Indirect calorimetry uses the oxygen consumption (VO2) and the carbon-dioxide production (VCO2) to compute the energy released by nutrients during oxidation. The total energy in kilo Joules (kJ) can be expressed terms of liters of O₂ consumed and liters of CO₂ produced (with a 2% error). An example energy calculation is shown below:

Carbohydrates: Glucose

C₆H₁₂O₆+6O₂→6CO₂+6H₂O+2812 kJ=1 g glucose+0.747 l O₂→0.747 l CO₂+0.6 g H₂O+15.7 kJ (Eq. 1)

Fats: Tripalmitin

C₅₁H₉₈O₆+72.5 O₂→51CO₂+49H₂O+32,036 kJ 1 g tripalmitin+2.011 l O₂→1.4161 CO₂+1.09 g H₂O+39.7 kJ (Eq. 2)

Protein: Beef protein (reaction for mammals)

4CH₃CH(NH₂)COOH+12O₂→2(NH₂)₂+10CO₂+10 H₂O+5223 kJ 1 g protein+0.992 l O₂→0.848 l CO₂+0.38 g H₂O+0.332 g Urea+18.4 kJ 1 g Urea=0.5 g of urinary nitrogen (Nᵤ) (Eq. 3)

Solving for the energy in terms of O₂, CO₂ and Nᵤ

Energy [kJ]=14.98 VO2 [1]+6.06 VCO2 [1]−7.42Nᵤ [g]For which the Nᵤ can be neglected incurring in a 2% error (Eq. 4)

Respiratory Quotient (RQ)=VCO2/VO2 (Eq. 5)

Thus, E=14.98 VO2+6.06 VCO2 and the can be expressed in power units (kilowatts or kW) TEE as follows: TEE=power (P)=Energy (E)/Time (t), where the measured O₂ and CO₂ are in liters per second. Since the TEE also depends on the temperature, the chamber is housed in a temperature controlled room inside a vivarium. A system of 24 metabolic chambers (e.g. CLAMS, Columbus Instruments, Columbus Ohio) is used. Such systems are known in the art. (e.g. CLAMS, Columbus Instruments, Columbus Ohio).

The Relative Lipolysis rate is measured. The relative rate of fat oxidation can be determined by the respiratory quotient (RQ) (Eq. 5). The RQ reflects the mixture of nutrients being oxidized. For example, as can be understood from the fat oxidation equation (Eq. 2), when fat is oxidized, more O2 is consumed than CO2 produced. As more fat is oxidized, the RQ ratio of CO2 produced: O2 consumed will decrease.

Food intake (Fᵢₙ) can be assessed. The metabolic chambers (e.g. CLAMS, Columbus Instruments, Columbus Ohio) have an integrated automatic food consumption monitoring system with an anti-spillage system. Such systems are known in the art (e.g. CLAMS, Columbus Instruments, Columbus Ohio). Food is replenished every 3 to 4 days.

d. Treatment of Appetite Suppressing Disorders by Deep Brain Stimulation

In cancer-cachexia, patients lose weight in an uncontrollable manner. Weight loss may be triggered by both a lower-than-required Fᵢₙ and a higher-than-needed TEE. As described above, the hypothalamic melanocortin circuit modulates Fᵢₙ and TEE. The hypothalamic melanocortin circuit has two cellular receptors, MCr3 and MCr4 and a very high concentration of these two cellular receptors are found in the VMH.

A DBS surgery is performed. After closing the abdominal incision from the tumor implantation surgery, animals in the SC and TR groups are placed in a stereotactic frame (e.g. Kopf instruments, Tujunga, Calif.). After securing the head of the animal to the frame, a midline scalp incision is made and skin and fascia are retracted to expose the midsagittal suture and lambda. The galea is cleaned from the skull to allow dental cement to adhere at the end of the procedure. Small holes are drilled bilaterally at the intended coordinates for placement of the electrodes. Stainless steel jeweler's screws are placed at 4 sites in the skull to provide anchoring for the dental cement, which holds the connector plug in place. Implanted materials are sterilized prior to surgery. A pin-type cable-connector is used to connect the implanted electrodes. Commercially available bipolar concentric platinum electrodes (e.g. SNEX 100, Kopf Instruments, Tujunga, Calif.) are used. After fixation of the cable connector, the acrylic is smoothed at the scalp-wound margins to prevent irritation. The incision wound margin is locally blocked with subcutaneously infiltrated Marcaine (0.5%) and partially closed with a single wound clip.

A stimulating circuit is used. A constant-current stimulation circuit to deliver a zero net charge via biphasic pulses is used. The circuit is carried by the animal on a modified commercially available rat jacket (e.g. Lomir Biomedical Malone, N.Y.). In order to keep it small and light weight a small battery will be used. The battery is changed once a week.

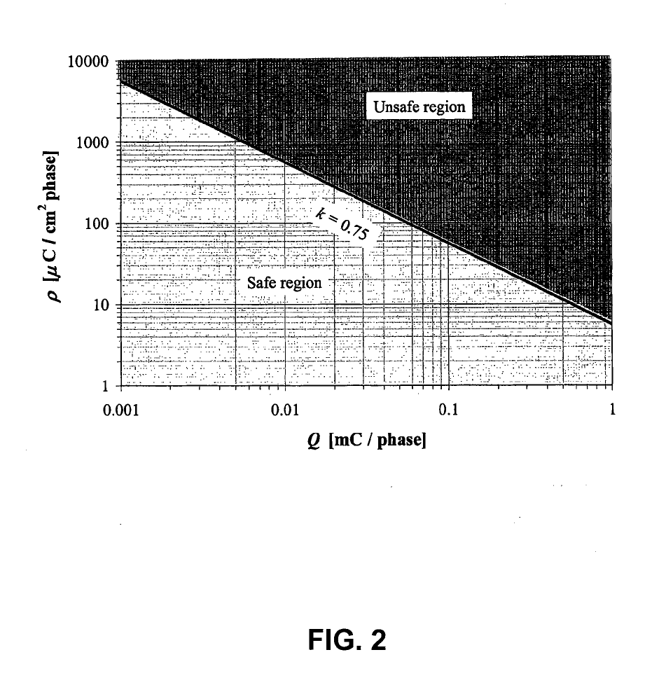

The neuromodulation is conducted based on the following protocol. Stimulation is commenced two weeks after implantation. Inhibitory stimulation is unilaterally or bilaterally delivered at very high frequency (10 kHz) via the implanted electrodes. Stimulating pulses are biphasic charged-balanced at constant current In some embodiments, stimulation amplitude is determined for each subject as the maximum amplitude at which the animal has no immediate obvious behavioral response. This threshold amplitude is established by progressively increasing the starting amplitude (10 μA) by 5 μA increments until a behavioral response is observed. The behavioral response is in the form of a transient change in behavior observed concurrently with the stimulation onset (i.e., does the animal “notice” the stimulation). The stimulation amplitude is defined as the current below the amplitude at which a behavioral response is observed. The amplitude remains below the damage threshold as shown in FIG. 2, which is a graph of stimulation amplitudes, and as determined by Equations 6 and 7. In other embodiments, the stimulation threshold is determined by monitoring oxygen consumption and determining whether there is a reduction in TEE.

An empirical relationship between charge/phase and charge density/phase was developed by Shannon et al., 1992 and is illustrated by the following equation:

(log(ρ)=k−log(Q)) (Eq. 6)

Eq. 6 was later corrected by McIntyre et al., 2001, for microelectrodes (see Eq. 7)

where Q is the charge per phase, ρ is the charge density per phase [μC/cm²], and k is an empirical factor in μC²/cm², safe→k<0.75 (McIntyre) (originally k<1.5 (Shannon)). (Eq. 7)

Because irreversible reactions can occur at high current densities, a safe and effective stimulation are determined based on an appropriate combination of current, charge, and charge density (see FIG. 2).

While the above-disclosed examples are described in terms of cachexia, a skilled artisan will understand that the examples, methods and apparatus disclosed herein may also be applied to anorexia, anorexia-nervosa and other appetite suppressing diseases and conditions. Accordingly, the disclosure should be considered to encompass other appetite suppressing and hypermetabolic diseases and conditions.

Although the present disclosure has been described with reference to preferred embodiments, persons skilled in the art will recognize that changes may be made in form and detail without departing from the spirit and scope of the disclosure.

What is claimed is:

1. A method of treating an appetite suppressing disorder or a disorder with an increased metabolic rate in a patient comprising:
identifying the brain structure that is subject to modulation in the patient; and
modulating the activity of one or more brain structures by applying electrical stimulation to one or more brain structures of a patient,
wherein the brain structure is chosen from the group consisting of the ventromedial hypothalamic nucleus, the perifornical region, the lateral hypothalamic area, the dorsomedial hypothalamic nucleus, the arcuate nucleus, and the paraventricular nucleus.

2. The method of claim 1 wherein identifying the brain structure further comprises administering to the patient an effective amount of an agonist or an antagonist of a cellular receptor of the brain structure.

3. The method of claim 1, wherein modulating the activity of a brain structure comprises modulating a system of the brain structure to treat an appetite suppressing disorder or a disorder with an increased metabolic rate.

4. The method of claim 2, wherein modulating the activity of a brain structure comprises modulating a system of the brain structure to treat an appetite suppressing disorder or a disorder with an increased metabolic rate.

5. The method of claim 3, wherein the system of the brain structure that is subject to modulation is chosen from the group consisting of the melanocortin system and the NPY system.

6. The method of claim 4, wherein the system of the brain structure that is subject to modulation is chosen from the group consisting of the melanocortin system and the NPY system.

7. The method of claim 1 further comprising imaging the brain structure that is subject to modulation.

8. The method of claim 1 further comprising modulating the activity of one or more brain structures by chemical stimulation by administering to the patient an effective amount of an agonist or an antagonist of a cellular receptor of the brain structure.

9. The method of claim 1, wherein the appetite suppressing disorder is chosen from the group consisting of cachexia and anorexia.

10. The method of claim 1 wherein the brain structure is the ventromedial hypothalamic nucleus.

11. The method of claim 6 wherein the brain structure is the ventromedial hypothalamic nucleus, the system is the melanocortin system and the cellular receptor is chosen from the group consisting of MCr3 and MCr4.

12. The method of claim 11 wherein the antagonist is selected from the group consisting of PG901 and MCLO129.

13. The method of claim 10 wherein the brain structure is modulated at high frequency stimulation or very high frequency stimulation.

14. The method of claim 11 wherein the brain structure is modulated at high frequency stimulation or very high frequency stimulation.

15. The method of claim 10 wherein identifying the brain structure that is subject to modulation further comprises administering glucose to the patient.

16. The method of claim 10 wherein the brain structure is a portion of the ventromedial hypothalamic nucleus selected from the group consisting of the dorsomedial portion of the ventromedial hypothalamic nucleus and the medial portion of the ventromedial hypothalamic nucleus.

17. The method of claim 11 wherein the brain structure is a portion of the ventromedial hypothalamic nucleus selected from the group consisting of the dorsomedial portion of the ventromedial hypothalamic nucleus and the medial portion of the ventromedial hypothalamic nucleus.

18. The method of claim 1 wherein the brain structure is the paraventricular nucleus.

19. The method of claim 6 wherein the brain structure is the paraventricular nucleus, the system is the melanocortin system and the cellular receptor is chosen from the group consisting of MCr3 and MCr4.

20. The method of claim 19 wherein the antagonist is selected from the group consisting of PG901 and MCLO129.

21. The method of claim 20 wherein the brain structure is modulated at a high frequency stimulation or a very high frequency stimulation.

22. The method of claim 18 wherein the brain structure is modulated at a high frequency stimulation or a very high frequency stimulation.

23. The method of claim 1 wherein the brain structure is the dorsomedial hypothalamic nucleus.

24. The method of claim 6 wherein the brain structure is the dorsomedial hypothalamic nucleus, the system is the NPY system and the cellular receptor is an NPY receptor.

25. The method of claim 24 wherein the agonist is selected from the group consisting of human/rat neuropeptide Y(2-36), dexamethasone[8] and N-acetyl[Leu 28, Leu 31] NPY (24-36).

26. The method of claim 24 wherein the brain structure is modulated at very low frequency stimulation, low frequency stimulation or medium frequency stimulation.

27. The method of claim 23 wherein the brain structure is modulated at very low frequency stimulation, low frequency stimulation or medium frequency stimulation.

28. The method of claim 1 wherein the brain structure is the lateral hypothalamic area.

29. The method of claim 6 wherein the brain structure is the lateral hypothalamic area.

30. The method of claim 29 wherein the cellular receptor is selected from the group consisting of 5-HT2C receptor and MOR receptor.

31. The method of claim 1, further comprising a step of fine-tuning, wherein the step of fine-tuning comprises monitoring at least one of oxygen consumption, energy expenditure, carbon dioxide production or respiratory quotient.

32. The method of claim 31, wherein the step of fine-tuning comprises monitoring oxygen consumption.

33. A kit comprising:
a neuromodulation device; and
instructions for using the neuromodulation device to modulate activity of a brain structure by applying electrical stimulation to one or more brain structures of a patient for treatment of an appetite suppressing disorder or a disorder with an increased metabolic rate.

34. The kit of claim 33 wherein the neuromodulation device comprises an implantable pulse generator, at least one lead and an extension.

35. The kit of claim 33 wherein the neuromodulation device is a deep brain stimulation system.

36. The kit of claim 33 wherein the appetite suppressing disorder is selected from the group consisting of cachexia and anorexia.

37. The kit of claim 33 wherein the one or more brain structures is selected from the group consisting of the ventromedial hypothalamic nucleus, the perifornical region, the lateral hypothalamic area, the dorsomedial hypothalamic nucleus, the arcuate nucleus, and the paraventricular nucleus.

38. The kit of claim 37 wherein modulating activity of a brain structure comprises modulating a system of the brain structure to treat an appetite suppressing disorder.

39. The kit of claim 38 wherein the system is selected from the group consisting of the melanocortin system and the NPY system.

40. The kit of claim 33 wherein the instructions further comprise identifying the brain structure to be modulated by administering to the patient an effective amount of an agonist or an antagonist of a cellular receptor of the brain structure.

41. The kit of claim 33 wherein the brain structure is the ventromedial hypothalamic nucleus.

42. The kit of claim 40 wherein the brain structure is the ventromedial hypothalamic nucleus, the system is the melanocortin system and the cellular receptor is chosen from the group consisting of MCr3 and MCr4.

43. The kit of claim 42 wherein the antagonist is selected from the group consisting of PG901 and MCLO129.

44. The kit of claim 43 wherein the brain structure is modulated at high frequency stimulation or very high frequency stimulation.

45. The kit of claim 41 wherein the brain structure is modulated at high frequency stimulation or very high frequency stimulation.

46. The kit of claim 41 wherein identifying the brain structure that is subject to modulation further comprises administering glucose to the patient.

47. The kit of claim 41 wherein the brain structure is a portion of the ventromedial hypothalamic nucleus selected from the group consisting of the dorsomedial portion of the ventromedial hypothalamic nucleus and the medial portion of the ventromedial hypothalamic nucleus.

48. The kit of claim 42 wherein the brain structure is a portion of the ventromedial hypothalamic nucleus selected from the group consisting of the dorsomedial portion of the ventromedial hypothalamic nucleus and the medial portion of the ventromedial hypothalamic nucleus.

49. The kit of claim 33 wherein the brain structure is the paraventricular nucleus.

50. The kit of claim 40 wherein the brain structure is the paraventricular nucleus, the system is the melanocortin system and the cellular receptor is chosen from the group consisting of MCr3 and MCr4.

51. The kit of claim 50 wherein the antagonist is selected from the group consisting of PG901 and MCLO129.

52. The kit of claim 49 wherein the brain structure is modulated at a high frequency stimulation or a very high frequency stimulation.

53. The kit of claim 50 wherein the brain structure is modulated at a high frequency stimulation or a very high frequency stimulation.

54. The kit of claim 33 wherein the brain structure is the dorsomedial hypothalamic nucleus.

55. The kit of claim 40 wherein the brain structure is the dorsomedial hypothalamic nucleus, the system is the NPY system and the cellular receptor is an NPY receptor.

56. The kit of claim 55 wherein the agonist is selected from the group consisting of human/rat neuropeptide Y(2-36), dexamethasone[8] and N-acetyl[Leu 28, Leu 31] NPY (24-36).

57. The kit of claim 55 wherein the brain structure is modulated at very low frequency stimulation, low frequency stimulation or medium frequency stimulation.

58. The kit of claim 54 wherein the brain structure is modulated at very low frequency stimulation, low frequency stimulation or medium frequency stimulation.

59. The kit of claim 33 wherein the brain structure is the lateral hypothalamic area.

60. The kit of claim 40 wherein the brain structure is the lateral hypothalamic area and the cellular receptor is selected from the group consisting of 5-HT2C receptor and MOR receptor.

61. The kit of claim 33, wherein the instructions further comprise a step of fine-tuning, wherein the step of fine-tuning comprises monitoring at least one of oxygen consumption, energy expenditure, carbon dioxide production or respiratory quotient.

62. The method of claim 31, wherein the step of fine-tuning comprises monitoring oxygen consumption.
